(19) United States
(12) Patent Application Publication
(10) Pub. No.: US 2024/0398659 A1
(43) Pub. Date: Dec. 5, 2024

(54) MALE MASTURBATION AID

(71) Applicant: Ary Co. Ltd., Tokyo (JP)

(72) Inventor: Tomofumi NARITA, Tokyo (JP)

(21) Appl. No.: 18/697,874

(22) PCT Filed: Apr. 19, 2022
(86) PCT No.: PCT/JP2022/018184

§ 371 Date: Apr. 2, 2024

(30) Foreign Application Priority Data
Oct. 29, 2021 (JP) 2021-177989

(51) Int. Cl. A61H 19/00

(52) U.S. Cl. CPC A61H 19/32; A61H 2201/1253; A61H 2201/1692

(57) ABSTRACT

A male masturbation aid having a flat bag shape, including a processed body including a sheet-like liner and an inner surface material in which unevenness regularly appears on the liner. The processed body is disposed on each of an upper surface and a lower surface inside the bag with the inner surface material facing inward. The aid prevents stickiness and discomfort after use, can be cleaned immediately without using tissue paper, can be folded and discarded and does not leak odor, and thus can be used every day without stress.

### TECHNICAL FIELD

The present invention relates to a technology of a tool for assisting masturbation of a male.

### BACKGROUND ART

Masturbation is an act of directly stimulating one's own genital without intercourse to obtain orgasm, and in many cases, the genitals are stimulated by hands, which is also referred to as manual masturbation, but a tool other than hands may be used.

Meanwhile, a plurality of technical proposals have been heretofore made for tools used for male masturbation, and for example, Patent Literature 1 proposes a sperm collector that is a means for collecting sperm by being worn on a penis and used, the sperm collector including a thin material such as a resin or rubber having elasticity (stretchability and flexibility) and easily elastically deformable.

### CITATION LIST

### Patent Literature

Patent Literature 1: Design Registration No. 1509127

### SUMMARY OF INVENTION

### Technical Problem

However, the above-described conventional technology has a problem that there is discomfort due to stickiness after use.

In addition, in the above-described conventional technology, it is necessary to use a tissue to remove dirt after use, and there is also a problem that it takes time and effort to use the tissue.

Therefore, in view of the above problems, an object of the present invention is to provide a male masturbation aid that prevents stickiness and discomfort after use, can be cleaned immediately without using tissue paper, can be folded and discarded and does not leak odor, and thus can be used every day without stress, and can be easily made into daily necessities because of a structure that can be mass-produced with an inexpensive material.

### Solution to Problem

One embodiment of the disclosed male masturbation aid is a male masturbation aid having a shape of a flat bag, including a processed body including a sheet-like liner and an inner surface material in which unevenness regularly appears on the liner, the processed body being disposed on each of an upper surface and a lower surface inside the bag with the inner surface material facing inward.

### Advantageous Effects of Invention

The disclosed male masturbation aid prevents stickiness and discomfort after use, can be cleaned immediately without using tissue paper, can be folded and discarded and does not leak odor, and thus can be used every day without stress, and can be easily made into daily necessities because of a structure that can be mass-produced with an inexpensive material.

### BRIEF DESCRIPTION OF DRAWINGS

FIG. 1 is a perspective view (part 1) of a male masturbation aid (first example) according to the present embodiment.

FIG. 2 is a perspective view (part 2) of the male masturbation aid (first example) according to the present embodiment.

FIG. 3 is a front view of the male masturbation aid (first example) according to the present embodiment.

FIG. 4 is a plan view of the male masturbation aid (first example) according to the present embodiment.

FIG. 5 is a bottom view of the male masturbation aid (first example) according to the present embodiment.

FIG. 6 is a right side view of the male masturbation aid (first example) according to the present embodiment.

FIG. 7 is a cross-sectional view taken along line A-A of the male masturbation aid (first example) according to the present embodiment.

FIG. 8 is a cross-sectional view taken along line B-B of the male masturbation aid (first example) according to the present embodiment.

FIG. 9 is a cross-sectional view taken along line C-C of the male masturbation aid (first example) according to the present embodiment.

FIG. 10 is a front view of a male masturbation aid (second example) according to the present embodiment.

FIG. 11 is a view illustrating a surface of a hollow portion of the male masturbation aid (second example) according to the present embodiment.

FIG. 12 is a cross-sectional view of the male masturbation aid (second example) according to the present embodiment.

### DESCRIPTION OF EMBODIMENTS

An embodiment of the present invention will be described with reference to the drawings.

### (Structure of Male Masturbation Aid According to Present Embodiment)

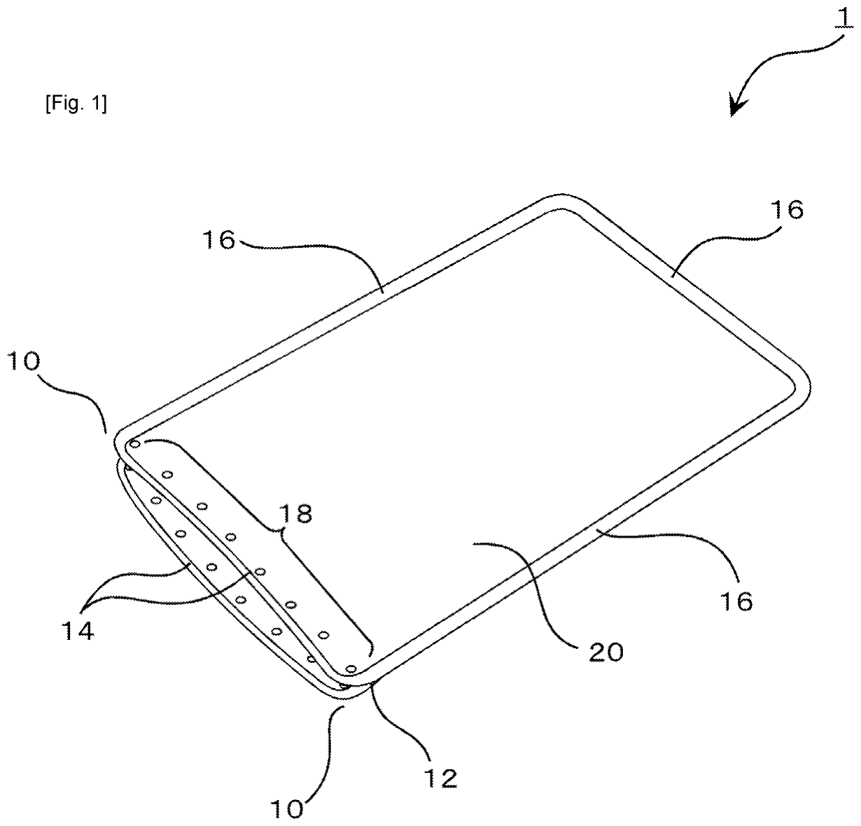

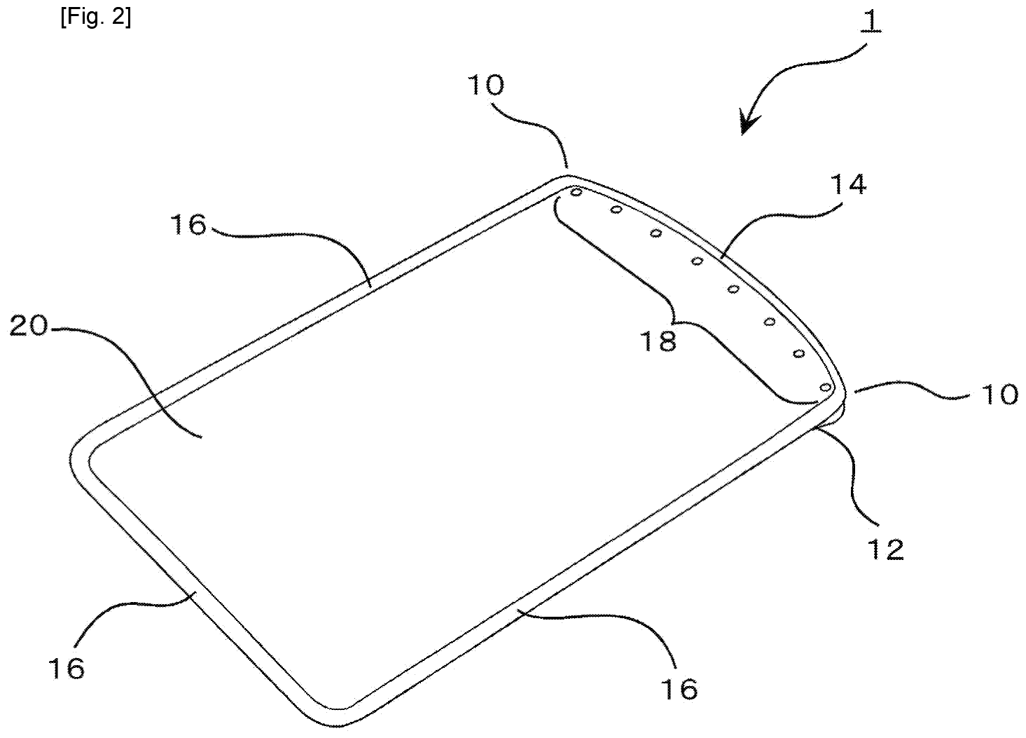

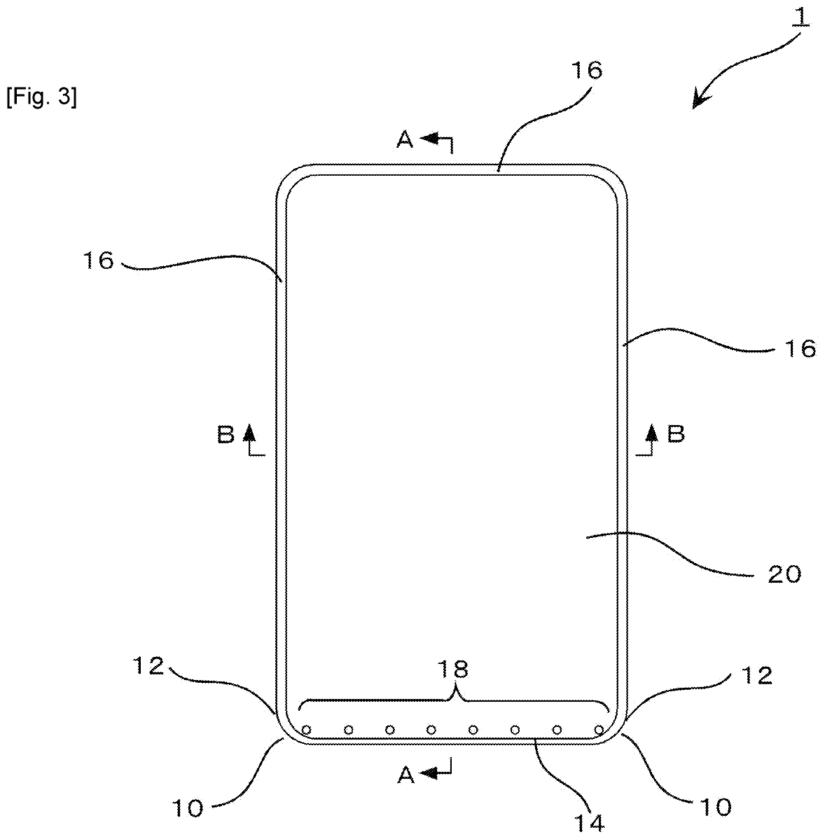

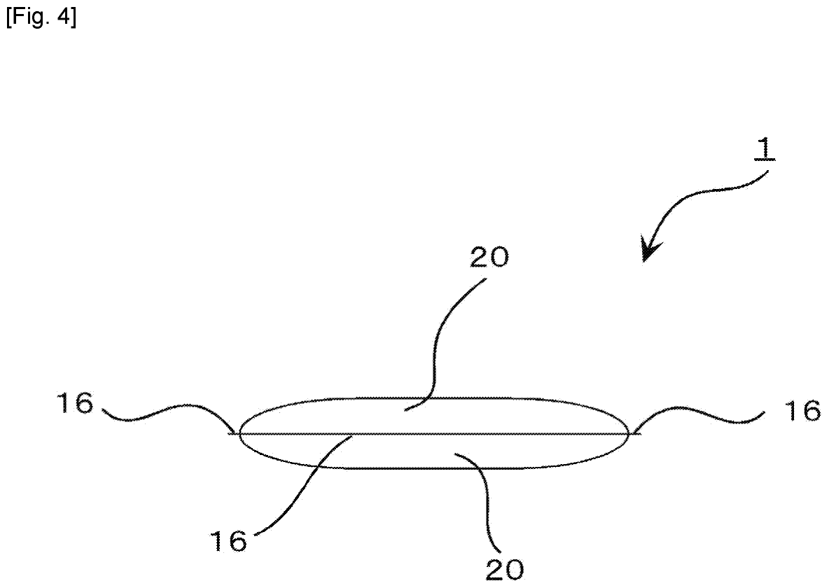

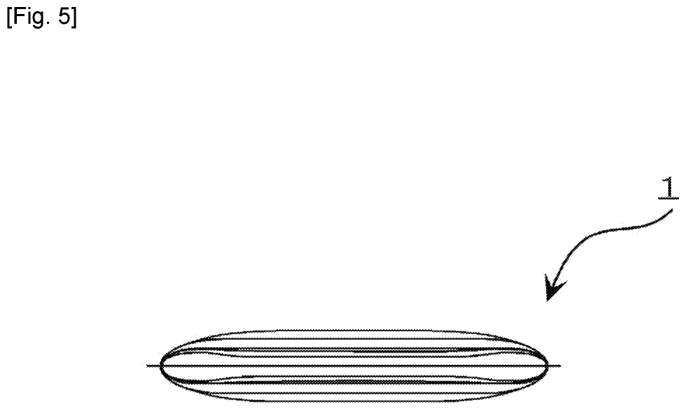

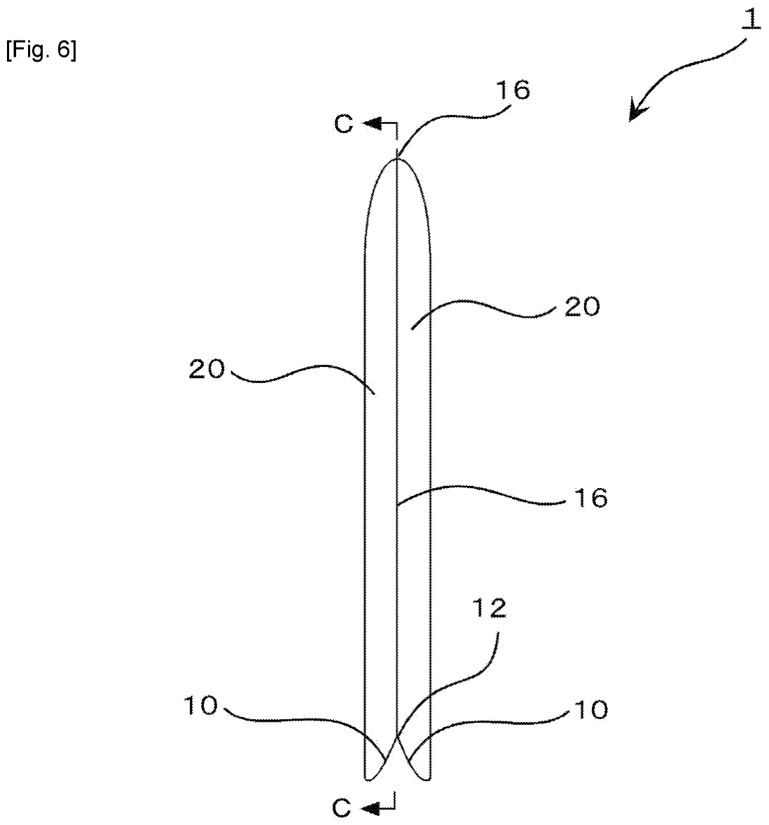

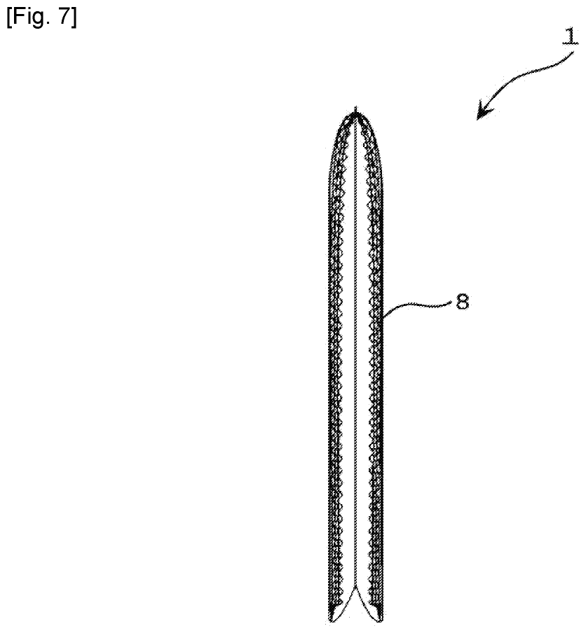

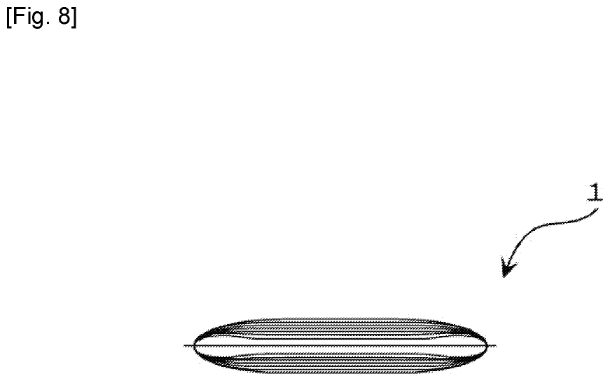

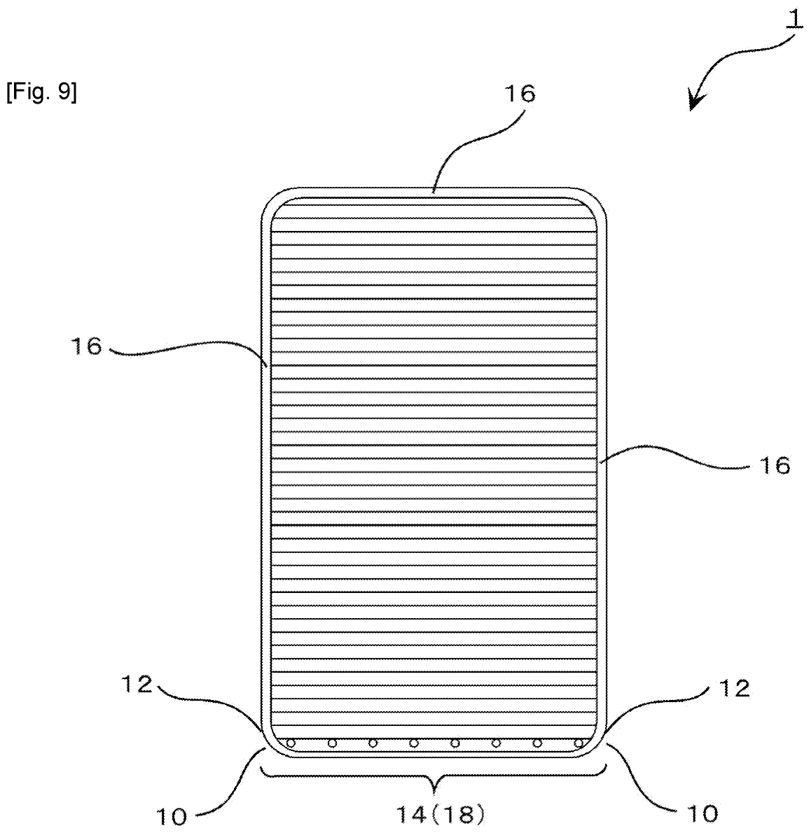

(1) First example: a structure of a male masturbation aid (hereinafter, simply referred to as “present aid”) 1 according to the present embodiment will be described with reference to FIGS. 1 to 9.

The present aid 1 is a tool that is mainly used by a male and helps a user to masturbate. That is, the present aid 1 is a tool for promoting male ejaculation. As illustrated in the drawings, the present aid 1 has a flat bag shape. The present aid 1 preferably has a size of, for example, about 15 cm wide×9 cm long, but the size may be appropriately changed. The present aid 1 is downsized, has a flat shape, and is easy to carry, and is not noticeable and does not cause embarrassment. That is, the present aid 1 is reduced in size, and portability and confidentiality are secured.

Each of the upper and lower surfaces of the bag 2 formed by the present aid 1 includes an outer surface material 20 disposed on the outer surface and a single-sided corrugated body 8 disposed inside the bag 2. The outer surface material 20 is preferably vinyl chloride, but may be other than vinyl chloride, and may be, for example, a silicon composition.

As illustrated in the drawings, the single-sided corrugated body 8 includes a sheet-like liner 4 and a wavy body 6 formed on the liner 4 so that a wavy form continuously appears. The single-sided corrugated body 8 is formed by stacking three layers formed of the liner 4 and the wavy body 6 in a state where the surface without the liner 4 faces the inside of the bag 2. Unlike a conventional cold male masturbation aid, it can provide a fluffy and warm feeling of use.

Further, the single-sided corrugated body 8 is disposed so that the wavy form formed by the wavy body 6 is continuous from an opening of the bag 2 toward a deep side. Note that the single-sided corrugated body 8 may be formed by changing the number of layers formed by the liner 4 and the wavy body 6 to one, two, or other numbers. As described above, a wavy form appears by performing corrugation processing on the nylon nonwoven fabric, and since the wavy form is unevenness continuing from an opening of a bag shape in a direction of a deep portion, the sense of use is enhanced by a moderate sense of resistance.

That is, since the inside of a bag portion of the present aid 1 is formed by stacking three layers of sheets having an uneven structure with small undulations, proper resistance and elasticity are exhibited.

Note that the liner 4 and the wavy body 6 are preferably formed of a nylon nonwoven fabric, but the material thereof may be other than the nylon nonwoven fabric and may be, for example, pulp, cotton, silicon, or the like.

In addition, the single-sided corrugated body 8 may have a form in which a nonwoven fabric coated with glycerin, saccharides, or the like is subjected to corrugation processing, and in this case, the single-sided corrugated body 8 has a better touch without using a lotion, and can eliminate stickiness and discomfort after use.

In addition, the single-sided corrugated body 8 may have a polka dot pattern (dot) shape, a lattice pattern shape, a wavy shape, or a linear uneven shape by embossing, three-dimensional processing, or the like.

As illustrated in the drawings, in the present aid 1, both left and right ends of the opening formed by the bag 2 are subjected to rounding 10 to round a corner. Further, the opening of the bag 2 is formed up to an end 12 of the portion subjected to the rounding 10. That is, both left and right ends of the opening of the bag 2 are rounded to remove a corner, and for example, have a shape of Y (when viewed from the side direction) of about 5 mm from the opening. The hardness of the portion in contact with the skin is suppressed, the opening is easily opened, and the feel of use is enhanced.

Since the present aid 1 has a flat bag shape, the single-sided corrugated body 8 and the outer surface material 20 are bonded in a band shape at a side portion and a bottom portion of the bag 2 (a periphery of the bag 2). Further, at the opening of the bag 2, the single-sided corrugated body 8 and the outer surface material 20 are bonded in a band shape on each of the upper and lower surfaces. This is to prevent the single-sided corrugated body 8 and the outer surface material 20 from being peeled off.

At this time, it is preferable that a width of the bonded portion between the single-sided corrugated body 8 and the outer surface material 20 is narrower around the opening of the bag 2 than a width of the side portion and the bottom portion of the bag 2.

Further, on a lower side of the bonded portion 14 around the opening of the bag 2, the single-sided corrugated body 8 and the outer surface material 20 are preferably bonded in a wavy line shape 18 along the opening end of the bag 2. Note that it is preferable that the single-sided corrugated body 8 and the outer surface material 20 are bonded by a thermocompression bonding method using a high-frequency seal.

As described above, the present aid 1 suppresses the hardness of the portion in contact with the skin, enhances the sense of use, and maintains the adhesive strength.

The present aid 1 may have a form including a tape member for folding the bag 2 in a lateral direction to change the size and holding the size, or folding the present aid 1 and holding the state. The tape member has a property of being easily attachable and detachable, and may be attached to the present aid 1 in a state of being attached thereto.

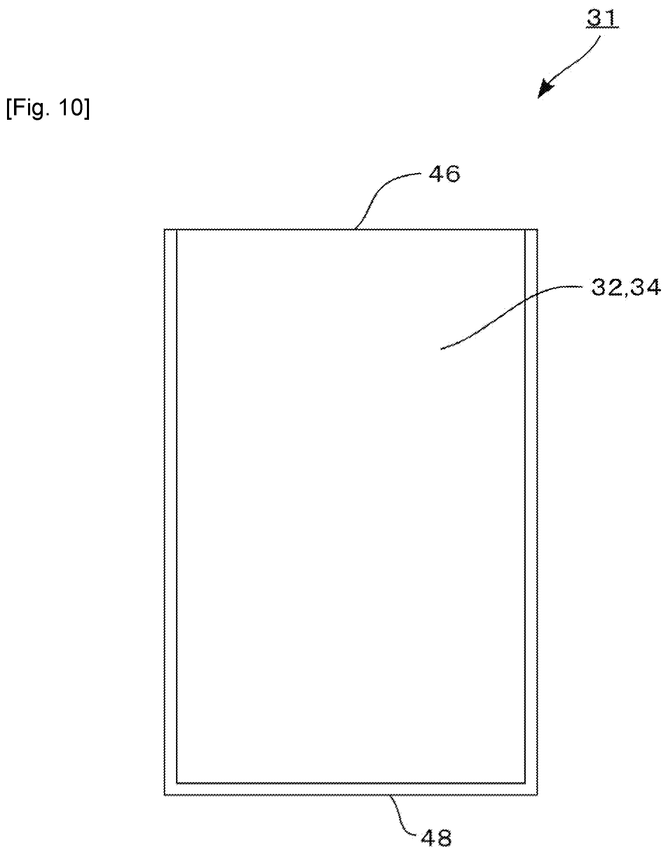

The present aid 1 may be provided with a lotion (an aqueous solution having viscosity and lubricity) (the lotion included in the package has a unique formulation to produce a quick-drying lotion, and since a nonwoven fabric is used as a material of the uneven portion, the lotion can be easily wiped off).
(2) Second example: a structure of a male masturbation aid (hereinafter, simply referred to as the “present aid”) 31 according to the present embodiment will be described with reference to FIGS. 10 to 12.

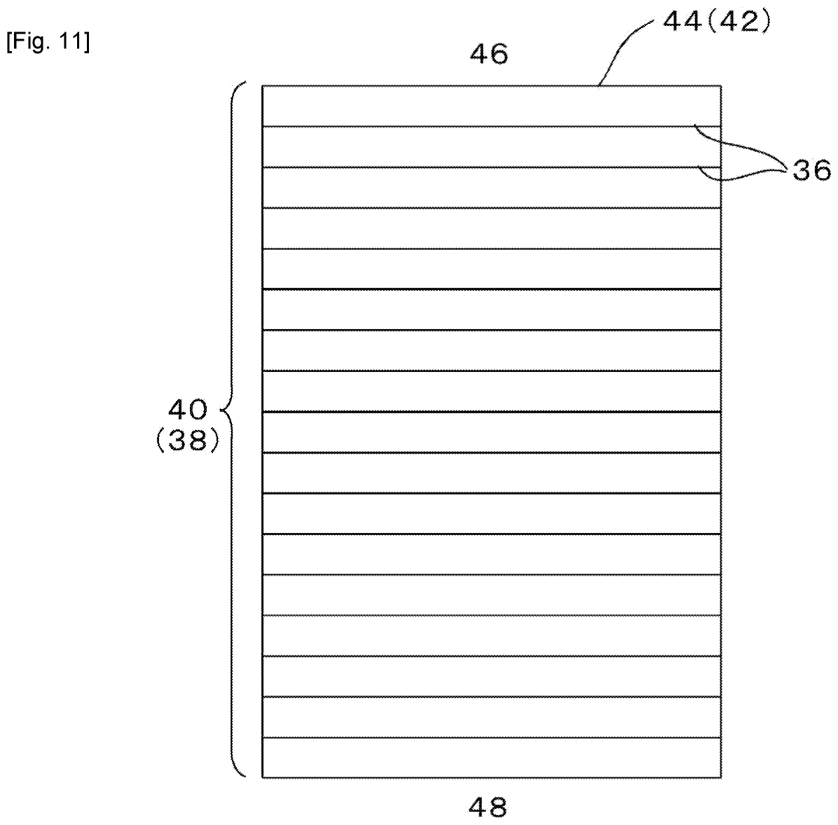

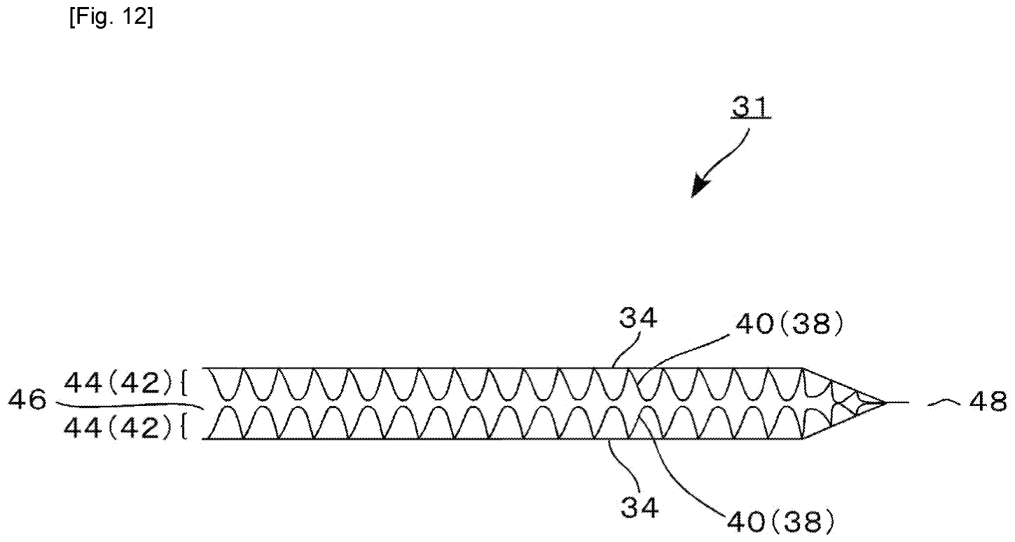

The present aid 31 is a tool that is mainly used by a male and assists a user in masturbation. That is, the present aid 31 is a tool for promoting male ejaculation. Here, FIG. 10 is a front view of the present aid 31, FIG. 11 is a view illustrating a surface of a hollow portion of the present aid 31, and FIG. 12 is a cross-sectional view of the present aid 31 taken along a line of an opening 46 of the bag 32 to a deep side 48.

As illustrated in FIGS. 10 to 12, the present aid 31 has a flat bag shape. The present aid 31 preferably has a size of, for example, about 15 cm×7 cm, but the size may be appropriately changed. The present aid 31 is downsized, has a flat shape, and is easy to carry, and is not noticeable and does not cause embarrassment. That is, the present aid 31 is miniaturized, and portability and confidentiality are secured.

(Upper and lower surfaces of) the bag 32 of the present aid 31 is constituted by a processed body 42. The processed body 42 includes a sheet-like liner 34 and an inner surface material 38 in which unevenness 36 regularly appears on the liner 34. Then, (the upper and lower surfaces of) the bag 32 of the present aid 31 is disposed with the inner surface material 38 facing inward.

Further, as illustrated in FIG. 11, (the upper and lower surfaces of) the bag 32 of the present aid 31 may be constituted by a single-sided corrugated body 44. The single-sided corrugated body 44 includes a sheet-like liner 34 and a wavy body 40 disposed on the liner 34 so that a wavy form continuously appears. As illustrated in FIGS. 11 and 12, (the upper and lower surfaces of) the bag 32 of the present aid 31 may have a form in which the single-sided corrugated body 44 is disposed in such an overlapping manner that the surface without the liner 34 faces inward and the wavy form is continuous from the opening 46 of the bag toward the deep side 48.

Note that in the single-sided corrugated body 44, the layer formed by the liner 34 and the wavy body 40 is preferably one layer, but the number of layers may be two, three, or more. Unlike a conventional cold male masturbation aid, it can provide a fluffy and warm feeling of use.

The wavy body 40 is preferably formed of a rayon mixed nonwoven fabric, but the material thereof is not limited as long as it is a nonwoven fabric. As described above, a wavy form appears by performing corrugation processing on the nonwoven fabric, and since the wavy form is unevenness continuing from the opening 46 of the bag shape in a direction of a deep portion 48, the sense of use is enhanced by a moderate sense of resistance.

That is, since the inside of the bag portion 32 of the present aid 31 is formed of a sheet having an uneven structure with small undulations, proper resistance and elasticity are exhibited.

The liner 34 is preferably formed of vinyl chloride or a nonwoven fabric which is a waterproof material, and the nonwoven fabric is preferably a rayon mixed nonwoven fabric, but it is not limited thereto. Note that the liner 34 forms the bag 32, that is, an outer portion of the present aid 31.

Based on the above structure, the present aid 31 prevents stickiness and discomfort after use, can be cleaned immediately without using tissue paper, can be folded and discarded and does not leak odor, and thus can be used every day without stress, and can be easily made into daily necessities because of a structure that can be mass-produced with an inexpensive material.

### (Method for Using Male Masturbation Aid According to Present Embodiment)

(1) First example: a method of using the present aid 1 will be described with reference to FIGS. 1 to 9. The present aid 1 is used by inserting the penis into the bag portion from the opening. Then, the present aid 1 can give an appropriate stimulation to the penis by repeating a sliding action between the inside of the bag portion and the penis inserted into the bag portion while the user grips the outside of the bag portion with the hand, and as a result, ejaculation is promoted.

First, after taking out the present aid 1 having a thin shape from a storage place, the user performs masturbation in a state where the opening of the bag 2 is formed and the penis is inserted into the bag 2 from the opening. Since the pressure can be adjusted by the user's own hand, the fit is good regardless of the size of the male genitalia.

Furthermore, the user adjusts the size of the bag 2 in the lateral direction according to the size of the male genitalia of the user with the tape member attached to the present aid 1, and thereby the sense of use can be further enhanced. Since the present aid 1 is has a thin material, the sense of feel when used can be enhanced by transmitting the body temperature and feeling the warmness of human skin.

After finishing the masturbation, the user folds the used present aid 1 and seals the opening with the attached tape member, so that the user can easily discard the aid 1 into a trash box or the like. By doing so, the user's hand is not dirty and clean, and the odor does not leak to the surroundings, so that the user can comfortably use it.

In addition, because the conventional male masturbation aid is expensive and can be structurally washed and reused, there are many users who actually reuse it and it is unsanitary, but the present aid 1 is inexpensive and structurally difficult to reuse, and can be used hygienically and cleanly as a disposable masturbation aid.

As described above, the present aid 1 prevents stickiness and discomfort after use, can be cleaned immediately without using tissue paper, can be folded and discarded and does not leak odor, and thus can be used every day without stress, and can be easily made into daily necessities because of a structure that can be mass-produced with an inexpensive material.
(2) Second example: a method of using the present aid 31 will be described with reference to FIGS. 10 to 12. The present aid 31 is used by inserting a penis into the bag portion 32 from the expanded opening 46. Then, the present aid 31 can give an appropriate stimulation to the penis by repeating a sliding action between the inside of the bag portion 32 and the penis inserted into the bag portion 32 while the user grips the outside of the bag portion 32 with the hand, and as a result, ejaculation is promoted.

First, after taking out the present aid 31 having a thin shape from a storage place, the user performs masturbation in a state where the opening 46 of the bag 32 is formed and the penis is inserted into the bag 32 from the opening 46. Since the pressure can be adjusted by the user's own hand, the fit is good regardless of the size of the male genitalia.

After finishing the masturbation, the user can fold the used present aid 31 and easily discard the present aid 31 into a trash can or the like. By doing so, the user's hand is not dirty and clean, and the odor does not leak to the surroundings, so that the user can comfortably use it.

In addition, because the conventional male masturbation aid is expensive and can be structurally washed and reused, there are many users who actually reuse it and it is unsanitary, but the present aid 31 is inexpensive and structurally difficult to reuse, and can be used hygienically and cleanly as a disposable masturbation aid.

As described above, the present aid 31 prevents stickiness and discomfort after use, can be cleaned immediately without using tissue paper, can be folded and discarded and does not leak odor, and thus can be used every day without stress, and can be easily made into daily necessities because of a structure that can be mass-produced with an inexpensive material.

Regarding the above embodiments, the following supplementary notes are further disclosed.

(Supplementary note 1) A male masturbation aid having a flat bag shape, in which

three layers of a single-sided corrugated body including a sheet-like liner and a wavy body disposed on the liner so that a wavy form continuously appears are disposed on each of the upper and lower surfaces inside the bag in such an overlapping manner that a surface without the liner faces inward and the wavy form is continuous from an opening of the bag toward a deep side;

both left and right ends of the opening of the bag are subjected to rounding to round a corner;

a width of a bonded portion of the single-sided corrugated body and the outer surface material of the bag on an outer periphery of the bag is narrower around the opening of the bag than in a side portion and a bottom portion of the bag, and on a lower side of the bonded portion on the outer periphery of the opening of the bag, the single-sided corrugated body and the outer surface material of the bag are bonded in a wavy line shape along an end of the opening of the bag; and

the opening of the bag is formed up to an end of the portion subjected to the rounding.

(Supplementary note 2) The male masturbation aid according to supplementary note 1, in which the single-sided corrugated body is formed of a nonwoven fabric.

(Supplementary note 3) The male masturbation aid according to supplementary note 2, in which the single-sided corrugated body is formed of a nylon nonwoven fabric, the outer surface material of the bag is formed of vinyl chloride, and the single-sided corrugated body and the outer surface material of the bag are bonded by a thermocompression bonding method.

Although the embodiments of the present invention have been described in detail above, the present invention is not limited to such specific embodiments, and various modifications and changes can be made within the scope of the gist of the present invention described in the claims.

### INDUSTRIAL APPLICABILITY

Distribution and sales method.
When a nonwoven fabric is used as a material and an existing manufacturing method such as corrugation processing is used in combination, mass production can be performed at low cost, and the nonwoven fabric can be easily used usually at low cost.
The present aid 1 is an individual packaging that can be suspended like a mask, and is compact in size with the packaging size of the mask, so that it is easy to purchase.
In some cases, the present aid 1 is packaged not in units of one piece but in a plurality of pieces together like a sanitary napkin, and has a shape that can be easily used daily as daily necessities.
The present aid 1 is packaged similarly to existing daily necessities to be easily purchased as daily necessities.

### REFERENCE SIGNS LIST

1 Male masturbation aid
2 Bag
4 Liner
6 Wavy body
8 Single-sided corrugated body
10 Rounded portion
12 End of rounded portion
14 Bonded portion around bag opening
16 Bonded portion at bag side portion and bottom portion
18 Bonded portion in wavy line shape
20 Outer surface material
31 Male masturbation aid
32 Bag
34 Liner
36 Unevenness on liner
38 Inner surface material
40 Wavy body
42 Processed body
44 Single-sided corrugated body
46 Opening of bag
48 Deep side of bag

1. A male masturbation aid having a shape of a flat bag, comprising:
a processed body including a sheet-like liner and an inner surface material in which unevenness regularly appears on the liner, the processed body being disposed on each of an upper surface and a lower surface inside the bag with the inner surface material facing inward.

2. The male masturbation aid according to claim 1, comprising:
a single-sided corrugated body including a sheet-like liner and a wavy body disposed on the liner so that a wavy form continuously appears, the single-sided corrugated body being disposed on each of the upper and lower surfaces inside the bag in such an overlapping manner that a surface without the liner faces inward and the wavy form is continuous from an opening of the bag toward a deep side.

3. The male masturbation aid according to claim 2, wherein the wavy body is formed of a nonwoven fabric, and the liner is formed of vinyl chloride or a nonwoven fabric.

4. The male masturbation aid according to claim 3, wherein the wavy body is formed of a rayon mixed nonwoven fabric, and the liner is formed of vinyl chloride or a rayon mixed nonwoven fabric.
